(19) United States
(12) Patent Application Publication
(10) Pub. No.: US 2009/0137989 A1
(43) Pub. Date: May 28, 2009

(54) Ophthalmic laser treatment device

(76) Inventor: Takuya Kataoka, Saitama (JP)

Correspondence Address: MORGAN LEWIS & BOCKIUS LLP, 1111 PENNSYLVANIA AVENUE NW, WASHINGTON, DC, 20004, US

(21) Appl. No.: 11/920,140

(22) PCT Filed: May 10, 2006
(86) PCT No.: PCT/JP2006/309377

§ 371 Date: Jan. 28, 2009

(30) Foreign Application Priority Data
May 10, 2005 (JP) 2005-137416
Dec. 13, 2005 (JP) 2005-358812

(51) Int. Cl. A61B 18/20

(52) U.S. Cl. 606/5

(57) ABSTRACT

A light-permeable member (lens 3) is provided to lead laser beams to an eyeball (2), and a spacer member (refrigerator forming portion 14) is provided to maintain a predetermined space (refrigerator chamber 13) between the light-permeable member (lens 3) and the eyeball (2). The eyeball (2) is cooled by introducing a fluid into the space (refrigerator chamber 13) so as to cool the eyeball (2) while applying the laser beams to the eyeball (2). The laser beams of higher intensity is applied with an occurrence of keratoedema reduced, while at the same time, preventing the complication which otherwise would be caused due to the heat generation of the eyeball (2).

### FIELD OF THE INVENTION

The invention relates to a device used for an ophthalmic laser treatment by making use of laser beams, and especially concerns to an ophthalmic laser treatment device used for an laser iridoctomy.

### BACKGROUND OF THE INVENTION

Upon performing a trans-corneal laser treatment in an ophthalmology, an cornea-contact type ocular lens has been used, so that laser beams released from a laser oscillator can reach inside the eyeball via the ocular lens and cornea in order to draw a therapeutical effect.

The laser treatment ocular lens is represented by an iridoctomy ocular lens as disclosed by Japanese Laid-open Patent Application No. 63-29639. The ocular lens is such that a laser-incident side lens and a laser-radiant side lens (eyeball-contact side lens) are accommodated into a lens holder, and prevent the outside light from entering inside the eyeball. When the laser is applied to the ocular lens with the lens placed on the eyeball, the lens enables an operator to bring the laser beams into focus on the iris. The focused laser beams increase an optical energy density so as to perforate the iris due to a heat generation of the increased optical energy density.

As mentioned above, the laser iridoctomy accompanies the heat generation, i.e., a somatic action due to the laser in order to perforate the iris by repetitively applying the laser beams to the iris. The laser beams bring the thermal influence on the cornea upon passing them through the cornea, while at the same time, bringing thermal effect on the iris and the aqueous humor due to the heat generation caused by the increased optical energy density given to the iris.

The thermal affect on the cornea, the iris and the aqueous humor often results in the complicated syndrome (complication) such as high ocular tension, bllous keratopathy and the like. Especially when the acute glaucoma develops, the complicated syndrome is likely to accompany due to the keratoedema.

The prior laser treatment ocular lens makes the lens in contact with the eyeball, thus blocking the heat accumulated in the eyeball from being released via the cornea. In order to avoid the complicated syndrome due to the heat generation, it is necessary to drop an intensity of the laser. However, the reduced intensity of the laser defies to draw the necessary therapeutical effect upon performing the iridoctomy.

Therefore, it is an object of the invention to overcome the above drawbacks, and provide an ophthalmic laser treatment device which is capable of cooling the eyeball to enhance the therapeutical effect by applying the laser beams of higher intensity, and using the laser beams with an occurrence of keratoedema reduced, while at the same time, preventing the complication due to the heat generation on the eyeball.

### DISCLOSURE OF THE INVENTION

### Means of Claim 3

In the ophthalmic laser treatment device of claim 3, a light-permeable member is provided to be in contact with an eyeball so as to lead laser beams to the eyeball. A fluid passage is provided through which a fluid flows to cool the eyeball. The fluid passage is located remote from the eyeball.

The structure is such that the eyeball is cooled through the light-permeable member which is in contact with the eyeball and cooled by the fluid. This makes it possible to cool the eyeball without making the fluid in direct contact with eyeball. This enables the operator to cool the eyeball without being influenced by the ingredients and streams of the fluid.

### Means of Claim 4

In the ophthalmic laser treatment device of claim 4, the fluid passage is located inside the light-permeable member. This makes it possible for the fluid to efficiently cool the light-permeable member because the fluid deprives the heat from inside the light-permeable member.

### Means of Claim 5

In the ophthalmic laser treatment device of claim 5, a transparent partition wall is provided to form a space between the light-permeable member and a laser oscillator provided to release the laser beams.

With the provision of the transparent partition wall, it is possible to avoid a dew condensation on the light-permeable member due to the evaporated vapor in the atmosphere. Under the absence of dew condensation, it is possible to favorably pass the laser beams through the light-permeable member without blocking the operator's visual field upon performing the laser treatment.

### Means of Claim 6

In the ophthalmic laser treatment device of claim 6, a transparent body is provided at one side of the light-permeable member, the one side being opposite to the eyeball, and a spacer is provided to form a space between the light-permeable member and the transparent body. The fluid passage is provided between the light-permeable member and the transparent body.

The structure is such that the fluid cools the light-permeable member by flowing the fluid over the one side of the light-permeable member opposite to the eyeball. This obviates the necessity of machining the light-permeable member to form a hollow space inside the light-permeable member to serve as a fluid passage.

### Means of Claim 7

In the ophthalmic laser treatment device of claim 7, the light-permeable member is formed by an elastic film of flexibility.

This enables the operator to attach the light-permeable member to the eyeball through an elastical contact therebetween, thus effectuating the light-permeable member which otherwise would incur injuries on the eyeball due to breakage when accidentally forcing the light-permeable member.

### Means of Claim 8

In the ophthalmic laser treatment device of claim 8, a fluid-supply member is provided to supply the fluid, and a control member is provided to functionally control the fluid-supply member. The control member adjusts a flow of the fluid so that the light-permeable member conforms its configuration to a configuration of the eyeball.

The structure is such that the control member brings the corneal contact wall (light-permeable member) snugly in contact with the eyeball without imposing a considerable burden on the eyeball.

### Means of Claim 9

In the ophthalmic laser treatment device of claim 9, a transparent partition wall is provided to form a space between the transparent body and a laser oscillator provided to release the laser beams.

With the provision of the transparent partition wall, it is possible to avoid a dew condensation on the light-permeable member due to the vapor evaporated in the atmosphere. Under the absence of dew condensation, it is possible to favorably permeate the laser beams through the transparent body without blocking the operator's visual field upon implementing the laser treatment.

### Means of Claim 10

In the ophthalmic laser treatment device of claim 10, the spacer and the light-permeable member are detachably mounted on the transparent body.

This enables the operator to form the fluid passage between the light-permeable member and the eyeball upon cooling the eyeball. Because the spacer and the light-permeable member are detachably arranged, it becomes possible to throw away the spacer and the light-permeable member each time after they are used once or several times, thus securing a good maintenance, high quality and sanitation preferable as a medical apparatus.

It is also possible to attach the spacer and the light-permeable member in various purposes to an existing ocular lens which is presently used widely to the ophthalmic laser treatment field.

### Means of Claim 11

In the ophthalmic laser treatment device of claim 11, a fluid-cooling member is provided to cool the fluid, and a control member is provided to functionally control the fluid-cooling member. The fluid-cooling member works to adjust so that the fluid substantially conforms its temperature to a target temperature.

This enables the operator to keep an appropriate fluid temperature which is variably required to cool the eyeball depending on the different laser-applying time and the cooling place.

### Means of Claim 12

In the ophthalmic laser treatment device of claim 12, a light-permeable member is provided to be in contact with an eyeball so as to introduce the laser beams to the eyeball. A Peltier's coupler is mounted on the light-permeable member. The light-permeable member is cooled by the Peltier's coupler so as to resultantly cool the eyeball.

This enables the operator to instantly cool the light-permeable member without using the fluid. The Peltier's coupler makes the light-permeable member immune to vibrations, as opposed to the case in which the fluid is used upon cooling the light-permeable member.

The Peltier's coupler has a good response to the temperature change so as to perform the temperature control with a high precision.

### Means of Claim 13

In the ophthalmic laser treatment device of claim 13, a reservoir is provided inside the light-permeable member to contain a fluid, and the Peltier's coupler works to cool the light-permeable member and the fluid contained inside the reservoir.

The fluid produces a convection in the fluid inside the reservoir to evenly cool the light-permeable member itself, compared to the case in which only the Peltier's coupler cools the light-permeable member.

The reservoir obviates the necessity of providing an inlet or outlet passage for supplying or exhausting the fluid which are otherwise required for the light-permeable member to circulate the fluid. This makes the light-permeable member into a simplified structure.

### Means of Claim 14

In the ophthalmic laser treatment device of claim 14, a transparent partition wall is provided to form a space between the light-permeable member and a laser oscillator provided to release the laser beams.

This makes it possible to avoid a dew condensation on the light-permeable member due to the vapor evaporated in the atmosphere. Under the absence of dew condensation, it is possible to favorably pass the laser beams through the transparent body without blocking the operator's visual field upon performing the laser treatment.

### Means of Claim 15

In the ophthalmic laser treatment device of claim 15, the light-permeable member is a lens member to collect the laser beams to bring the laser beams into focus on the eyeball.

This enables the operator to bring the laser beams into focus on desired locations of the eyeball, while at the same time, enhancing the density level of the laser beams.

### Means of Claim 16

In the ophthalmic laser treatment device of claim 16, a focusing member is provided to bring the laser beams into focus on any position of the eyeball by refracting or reflecting the laser beams incident on the light-permeable member.

This enables the operator to bring the laser beams into focus on desired locations of the eyeball, while at the same time, enhancing the density level of the laser beams.

### BRIEF DESCRIPTION OF THE DRAWINGS

FIG. 1 is a longitudinal cross sectional view of an ophthalmic laser treatment device according to a first embodiment of the invention;

FIG. 2 is a longitudinal cross sectional view of an ophthalmic laser treatment device according to a modification form of the first embodiment of the invention;

FIG. 3 is a longitudinal cross sectional view of an ophthalmic laser treatment device according to other modification form of the first embodiment of the invention;

FIG. 4 is a perspective view of an ophthalmic laser treatment device according to a second embodiment of the invention but partly sectioned;

FIG. 5 is a longitudinal cross sectional view of an ophthalmic laser treatment device according to a third embodiment of the invention;

FIG. 6 is a longitudinal cross sectional view of an ophthalmic laser treatment device according to a fourth embodiment of the invention;

FIG. 7 is a longitudinal cross sectional view of an ophthalmic laser treatment device according to a fifth embodiment of the invention;

FIG. 8 is a longitudinal cross sectional view of an ophthalmic laser treatment device according to a sixth embodiment of the invention;

FIG. 9(a) is a longitudinal cross sectional view of an ophthalmic laser treatment device according to a seventh embodiment of the invention; and

FIG. 9(b) is a side elevational view of the ophthalmic laser treatment device observed from a laser oscillator according to the seventh embodiment of the invention.

### DESCRIPTION OF NUMERALS

1 ophthalmic laser treatment device
2 eyeball
3 lens (light-permeable member, transparent body and focusing member)
4 lens holder
5 partition wall
10 holder cylinder
20 tank
21 pump (fluid-supply member)
22 computer (control member)
30 inner fluid passage (fluid passage)
40 outer side fluid passage (fluid passage)
50 flow passage
60 flow passage
61 cornea-contact wall (light-permeable member)
62 fluid-passage forming portion (spacer)
65 refrigerator device (fluid-cooling member)
80 Peltier's coupler
90 reservoir

### BEST MODE FOR CARRYING OUT THE INVENTION

According the present invention, an ophthalmic laser treatment device enables an operator to manipulate laser beams while cooling an eyeball with an occurrence of keratioedema substantially reduced. For this purpose, a light-permeable member is provided to be in contact with an eyeball so as to lead laser beams to the eyeball. A fluid passage is provided, through which a fluid flows to cool the eyeball. The fluid passage is located remote from the eyeball.

### First Embodiment of the Invention

### Structure of First Embodiment

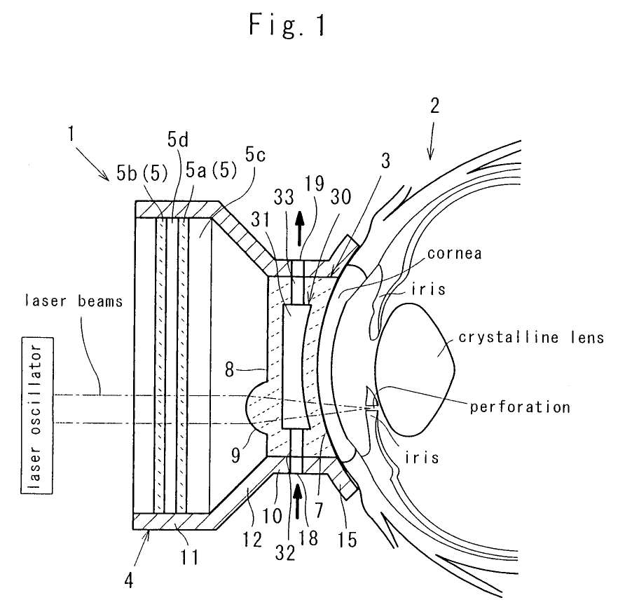

Referring to FIG. 1, described is an ophthalmic laser treatment device 1 according to a first embodiment of the invention. FIG. 1 shows a schematic view of the ophthalmic laser treatment device 1 which is placed in contact with an eyeball 2. In FIG. 1, an up-to-down line from top to bottom accords to a gravitational direction, and the left side of the drawing is where a laser oscillator is located. The right side of the drawing is where the eyeball resides, an upper side is a parietal side and the lower side is a maxillary side.

The ophthalmic laser treatment device 1 introduces laser beams from the laser oscillator to the eyeball 2 of a subject patient, and having a lens 3 which collects the laser beams and brings the laser beams into focus on the eyeball 2. In ophthalmic laser treatment device 1, a lens holder 4 is provided to structurally support the lens 3. A transparent partition wall 5 is provided to form a hermetic space in cooperation with the lens 3. In the first embodiment of the invention, the lens 3 acts as the light-permeable member which comes in contact with the eyeball 2 to introduce the laser beams to the eyeball 2.

The lens 3 is formed into a discus-like configuration from a transparent material, and having a concave surface side 7 shaped in accordance with an outer surface of the eyeball 2. The lens 3 has an incident surface 8 as a convex side portion 9, on which the laser beams is incident from the laser oscillator. The convex side portion 9 of the lens 3 projects toward the laser oscillator to refract the laser beams to bring the laser beams into focus on the iris in which the laser beams increase its density.

The lens holder 4, according to the invention, is in the shape of a cylinder, and having a holder cylinder 10, to an inner circumferential side of which the lens 3 is hermetically attached. The lens 3 has a cylindrical portion 11 at one end which opposes the laser oscillator. The cylindrical portion 11 is diametrically greater than the lens 3. Between the cylindrical portion 11 and the holder cylinder 10, a tapered cylinder 12 is provided which diametrically increases progressively as approaching the cylindrical portion 11. The holder cylinder 10, the cylindrical portion 11 and the tapered cylinder 12 are integrally formed to define the lens holder 4 by means of a synthetic resin.

In the lens holder 4, a peripheral open end of the holder cylinder 10 defines the engagement portion 15 to be in contact with the eyeball 2. The engagement portion 15 shapes its outer surface and the concave surface side 7 into a concave configuration in accordance with the outer surface of the eyeball 2.

In the ophthalmic laser treatment device 1, an inner portion of the lens 3 is hollowed out to form an inner passage 30. The inner passage 30 has a columnar hollow chamber 31, an inlet passage 32 and an outlet passage 33. The hollow chamber 31 is provided in the middle of the lens 3. The inlet passage 32, which is provided in the jowl side, is in communication with the hollow chamber 31. The outlet passage 33, which is provided in the parietal side, is in communication with the hollow chamber 31. At the side of the jowl, the holder cylinder 10 defines the inlet hole 18, and forming the outlet hole 19 at the parietal side of the subject patient. The inlet hole 18 is in communication with the inlet passage 32, and the outlet hole 19 is in communication with the outlet passage 33.

The tank supplies the fluid from the inlet hole 18 and discharged out of the outlet hole 19 via the inlet pas sage 32, the hollow chamber 31 and the outlet passage 33.

The ophthalmic laser treatment device 1 further has a fluid-supply member and a control member which functionally controls the fluid-supply member. The fluid-supply member works as a pump which supplies the fluid from a tank via the inlet hole 18 to the refrigerator chamber 13. The tank is connected to the inlet hole 18 as a fluid supply source. The control member employs a computer as a well-known measure to functionally control the pump. By adjusting a suction speed of the pump, it is possible to control an amount of the fluid flowing to the refrigerator chamber 13. By detecting the temperature of the fluid in the tank, and detecting the temperature of the fluid exhausted from the outlet 19, it is possible to adjust the fluid speed in accordance with the thermal rise from the latter temperature to the former temperature.

The partition wall 5 is formed by a transparent material, and located in registration with the lens 3 at the opposite side of the eyeball 2. The partition wall 5 is hermetically fixed to an inner side of the cylindrical portion 11. The partition wall 5 has two vitreous plates (first head wall 5a and second head wall 5b) located in parallel.

The first head wall 5a forms a first space 5c together with the lens 3, and the second head wall 5 forms a second space 5d together with the first head wall 5a.

### Therapeutical Method According to the First Embodiment of the Invention

Upon performing the iridoctomy by the ophthalmic laser treatment device 1, the laser beams are applied to the lens 3 with the lens 3 and the concave surface side 7 placed on the eyeball 2, the lens enables an operator to bring the laser beams into focus on the iris.

The focused laser beams increase an optical energy density so as to perforate the iris. While applying the laser beams to the lens 3, the fluid (e.g., cooled water) is supplied intermittently through the inlet hole 18. The lens 3 introduces the laser beams through the fluid into the eyeball 2. The fluid flows along the inner passage 30 and deprives the heat of the inner passage 30 to cool the lens 3. The cooled lens 3 deprives the heat of the eyeball 2 so as to perform the laser treatment while cooling the eyeball 2.

In accordance with the heat generation on the eyeball 2 due to the laser beams, the control member adjusts to increase the suction speed of the pump 21 which supplies the fluid to the refrigerator chamber 13, thus increasing the amount of the fluid flowing to the refrigerator chamber 13, so as to therapeutically perform the laser treatment while cooling the eyeball 2.

### Advantages Derived from the First Embodiment of the Invention

In the ophthalmic laser treatment device 1, with the inner passage 30 defined hollow in the inner portion of the lens 3, the operator enables the fluid to flow through the inner passage 30 to cool the lens 3. The cooled lens 3 deprives the heat of the eyeball 2 to resultantly cool the eyeball 2.

This makes it possible to cool the eyeball 2 without making the fluid in direct contact with eyeball 2. This enables the operator to cool the eyeball 2 without being influenced by the ingredients and streams of the fluid on the eyeball 2 upon therapeutically performing the laser treatment.

With the eyeball 2 cooled while applying the laser beams to the eyeball 2, it is possible to prevent the complication which otherwise would be caused by the temperature rise of the eyeball 2 due to the laser beam application. The inner passage 30 enables the operator to efficiently cool the lens 3 since the lens 3 permits the fluid to flow through an interior of the lens 3.

The holder cylinder 10 has the outlet hole 19 at the parietal side in the direction opposite to the gravitational direction. If the fluid appears forms therein, the refrigerator forming portion 14 collects the foams at the outlet hole 19 and exhausts the foams out of the refrigerator chamber 13 without retaining them in the fluid. With no foams retained in the fluid, it is possible to perform the laser treatment without blocking the laser beam path and the visual field of the operator.

By detecting the temperature of the fluid in the tank, and detecting the temperature of the fluid exhausted from the outlet 19, it is possible to adjust the fluid speed by controlling the suction speed of the pump based on the thermal rise from the latter temperature to the former temperature. This makes it possible to perform the laser treatment while keeping the eyeball 2 cooled substantially at a constant temperature.

When the fluid is supplied to the refrigerator chamber 13, the lens 3 reduces its temperature than the atmospheric temperature. With the head walls 5a, 5b hermetically sealed within the cylindrical portion 11 between the lens 3 and the laser oscillator to form the first space 5c and the second space 5d, it is possible for the spaces 5c, 5d to enhance an adiabatic effect so as to prevent the dew condensation on the lens 3, thus preventing the laser beam path and the visual field from being blocked on the lens 3. It is to be noted that one of the head walls 5a, 5b may be omitted upon effectuating the adiabatic effect. By making the spaces 5c, 5d vacuous, it is possible to increasingly improve the adiabatic effect. The transparent partition wall 5 prevents the laser beam path and the visual field from being blocked on the lens 3.

### Modification Form of the First Embodiment of the Invention

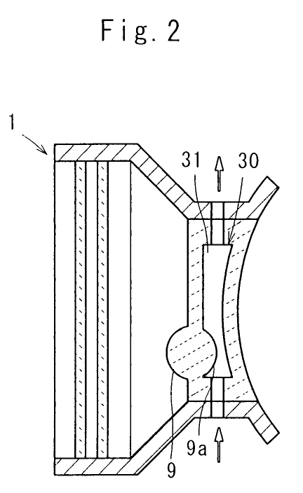

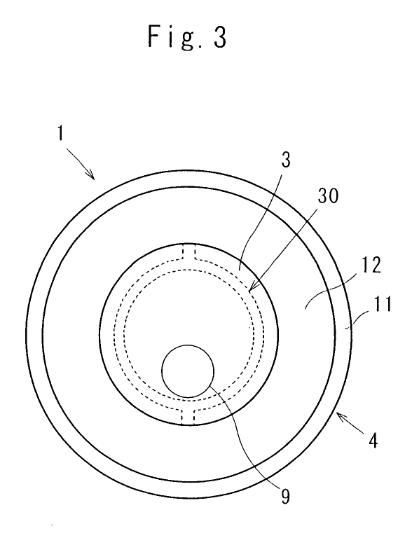

In the modification form of the first embodiment of the invention as shown in FIG. 2, a lower side of the convex portion 9 of the lens 3 has a convex surface 9a projected into the hollow chamber 31 to form a convexo-convex lens. Instead of the columnar hollow chamber 31, an annular hollow chamber may be provided as shown in FIG. 3. Instead of hollowing out the inner portion of the lens 3 upon forming the hollow chamber 31, two dish-shaped transparent members may be joined in a face-to-face fashion to define the hollow chamber 31 inside the joined dish-like members.

It is to be noted that the fluid is permeable to the laser beams in the first embodiment of the invention, however, the fluid may be impermeable to the laser beams when the inner passage 30 is provided out of a permeative path of the laser beams.

### Second Embodiment of the Invention

### Structure of Second Embodiment

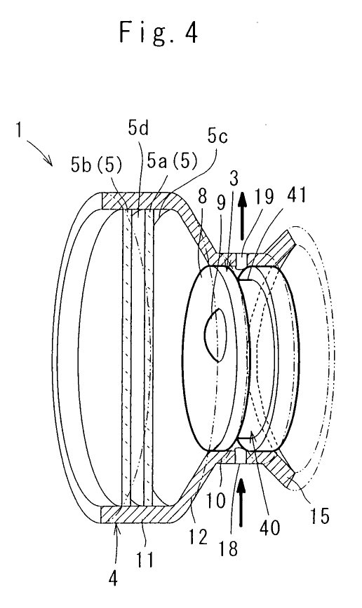

FIG. 4 shows a second embodiment of the invention which mainly describes the structure other than the first embodiment of the invention. The contact condition between the eyeball 2 and the lens 3 in the second embodiment of the invention, is substantially identical to the contact condition in the first embodiment of the invention.

In the second embodiment of the invention, an outer side passage 40 is provided to flow the fluid along an outer circumference of the lens 3.

Along an entire length of the outer circumference of the lens 3, a circumferential groove 41 is provided. By liquid-tightly covering the circumferential groove 41 by the inner surface of the holder cylinder 10, the outer side passage 40 is defined at the outer circumference of the lens 3. The holder cylinder 10 provides the inlet hole 18 and the outlet hole 19. The inlet hole 18 permits the entrance of the fluid, and the outlet hole 19 permits the exhaust of the fluid.

### Therapeutical Method According to the Second Embodiment of the Invention

With the lens 3 placed on the eyeball 2, the laser beams are released against the lens 3. During the release of the laser beams, the tank intermittently supplies the fluid to the outer side passage 40 through the inlet hole 18, thus making the fluid flow along the outer side passage 40 to deprive the heat of the lens 3 to cool the lens 3. The cooled lens 3 deprives the heat of the eyeball 2 during the release of the laser beams, so as to perform the laser treatment while cooling the eyeball 2.

### Advantages Derived from the Second Embodiment of the Invention

In the ophthalmic laser treatment device 1 according to the second embodiment of the invention, the outer side passage 40 enables the operator to flow the fluid to cool the lens 3 without using a highly technical procedure of hollowing out the inner portion of the lens 3.

With the eyeball 2 cooled while applying the laser beams to the eyeball 2, it is possible to prevent the complication which would be otherwise caused by the temperature rise of the eyeball 2 due to the laser beam application. When highly intensified laser beams are employed to enhance therapeutical effects, the fluid flow enables the operator to cool the lens 3 so as to avoid the complication.

As a modification form of the second embodiment of the invention, instead of providing the circumferential groove 41 on the outer periphery of the lens 3, a concave groove may be formed on an inner surface of the holder cylinder 10 in the circumferential direction to define the outer side passage 40. Otherwise, instead of the holder cylinder 10, an annular pipe may be provided to flow the fluid therethrough so as to define the outer side passage 40. As an alternative, a discrete member may be added around the holder cylinder 10 to provide the outer side passage 40 on an outer surface of the holder cylinder 10.

### Third Embodiment of the Invention

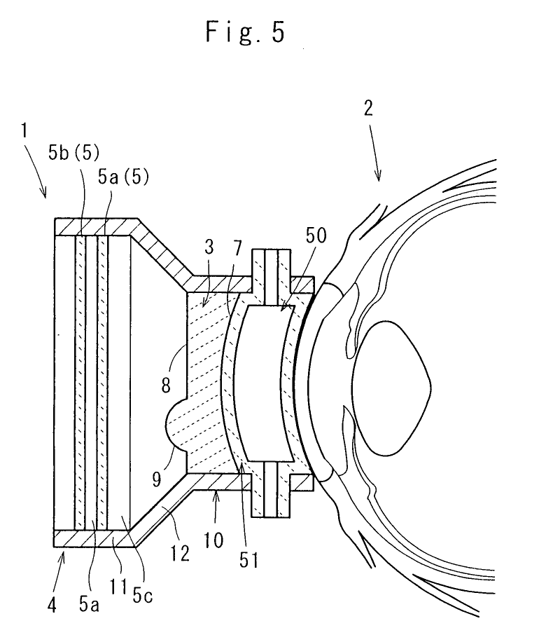

FIG. 5 shows a third embodiment of the invention in which the ophthalmic laser treatment device 1 is applied to the eyeball 2. In FIG. 5 which is a schematic view of the ophthalmic laser treatment device 1, the light-permeable member is a refrigerant vessel 51, an inner portion of which is defined as a flow passage 50. The refrigerant vessel 51 is placed between the lens 3 and the eyeball 2. The lens 3 acts as a focusing member which refracts the laser beams incident on the refrigerant vessel 51 to bring the laser beams into focus on any location of the eyeball 2.

The refrigerant vessel 51 is formed by a transparent light-permeable material. The refrigerant vessel 51 has an ocular contact side shaped into a concave configuration to correspond to an outer surface of the eyeball 2. The refrigerant vessel 51 has a lens contact side shaped into a convex configuration to correspond to the concave surface side 7 of the lens 3.

The refrigerant vessel 51 is detachably mounted on the lens 3 and the holder cylinder 10. Because the refrigerant vessel 51 is detachably arranged, it becomes possible to throw away the refrigerant vessel 51 each time after it is used once or several times, thus securing a good maintenance, high quality and sanitation preferable as a medical apparatus.

### Fourth Embodiment of the Invention

### Structure of Fourth Embodiment

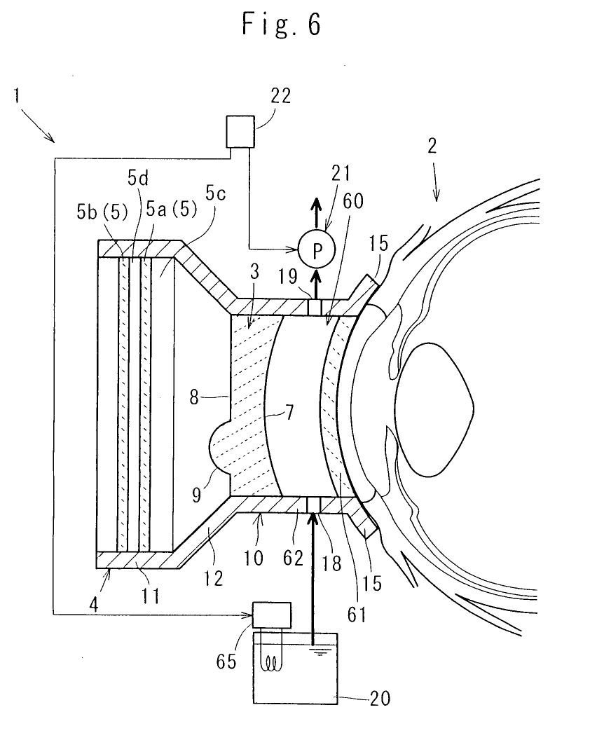

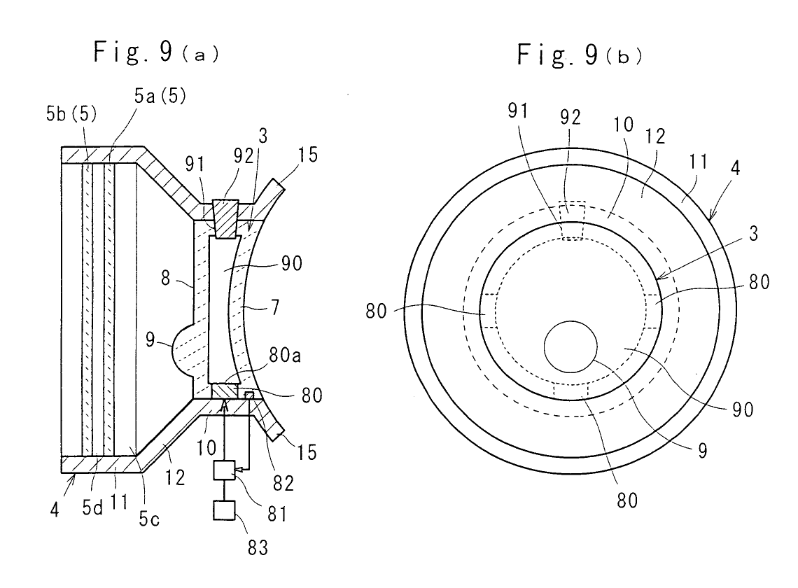

FIG. 9 shows a fourth embodiment of the invention in which the ophthalmic laser treatment device 1 is applied to the eyeball 2. In FIG. 6, the up-to-down line from top to bottom accords to the gravitational direction, and the left side of the drawing is where the laser oscillator is located. The right side of the drawing is where the eyeball resides, the upper side is the parietal side and the lower side is the maxillary side.

In the sixth embodiment of the invention, the ophthalmic laser treatment device 1 has the lens 3, the lens holder 4 and the transparent partition 5 which forms the hermetical space in cooperation with the lens 3. The ophthalmic laser treatment device 1 has a cornea-contact wall 61 between the lens 3 and the eyeball 2 to provide a flow passage 60. The cornea-contact wall 61 serves as the light-permeable member which is in contact with the eyeball 2 to lead the laser beams to the eyeball 2. The lens 3 acts as a transparent body which forms a flow passage 60 between the lens 3 and the cornea-contact wall 61. The lens 3 is also a focusing member which refracts the laser beams incident on the cornea-contact wall 61 to bring the laser beams into focus on any location of the eyeball 2.

According to the lens holder 4 of the fourth embodiment of the invention, there is fixedly provided the lens 3 at the inner surface of the holder cylinder 10. A fluid-passage forming portion 62 is provided as a spacer at an intermediary portion nearer to the eyeball 2 than the concave surface side 7 between the lens 3 and the cornea-contact wall 61 to define the flow passage 60.

In order to supply the fluid to the flow passage 60, the fluid-passage forming portion 62 defines the inlet hole 18 at the side of the jowl, and forming the outlet hole 19 at the parietal side.

The lens holder 4 is open at an ocular portion in which the fluid-passage forming portion 62 opposes the eyeball 2. The fluid-passage forming portion 62 is shaped into a skirt-like configuration at an open end which opposes the eyeball 2, and having the engagement portion 15 which is placed in contact with the eyeball 2 when in use.

The cornea-contact wall 61 is liquid-tightly fixed to an inner surface of the fluid-passage forming portion 62 at the side of the eyeball 2 to be in contact with the eyeball 2. The cornea-contact wall 61 is formed by an elastic film (e.g., polymer film) of flexibility.

The partition wall 5 is formed by a transparent material, and located in registration with the lens 3 at the opposite side of the eyeball 2. The partition wall 5 is hermetically fixed to an inner side of the cylindrical portion 11.

The partition wall 5 has two vitreous plates (first head wall 5a and second head wall 5b) located in parallel. The first head wall 5a forms a first space 5c in cooperation with the lens 3, and the second head wall 5 forms a second space 5d in cooperation with the first head wall 5a.

The ophthalmic laser treatment device 1 further has the fluid-supply member, a fluid-cooling member and a control member which controls the fluid-supply member and the fluid-cooling member. The fluid-cooling member works as a refrigerant device 65 which is placed in the tank 20 to fall the fluid temperature in the tank 20.

The fluid-supply member works as a pump 21 to supply the fluid from the tank 20 via the inlet hole 18 to the flow passage 60. The tank 20 is connected to the inlet hole 18. The control member employs the computer 22 as a well-known measure to control the pump 21 and the refrigerant device 65.

By adjusting a suction speed of the pump 21, it is possible to control an amount of the fluid running through the flow passage 60 so as to adjust an inner pressure of the flow passage 60. Because the cornea-contact wall 61 is the elastic film, the inner pressure of the flow passage 60 deforms the cornea-contact wall 61 at a certain curvature. A constant inner pressure of the flow passage 60 maintains the cornea-contact wall 61 at the predetermined curvature. By calculating a relationship between the inner pressure of the flow passage 60 and the curvature of the cornea-contact wall 61, it becomes possible to control the suction speed of the pump 21 to change the inner pressure so that the cornea-contact wall 61 is adjusted at the curvature suited to the curvature of the eyeball 2. The temperature of the fluid cooled by the refrigerant device 65 is controlled with the temperature of the fluid exhausted from the outlet hole 19 as a target temperature.

### Therapeutical Method According to the Fourth Embodiment of the Invention

Upon performing the laser treatment, the laser beams are applied to the lens 3 with the cornea-contact wall 61 placed on the eyeball 2 via an ophthalmic lubricant in contact with the eyeball 2. During the release of the laser beams, the pump 21 supplies the fluid intermittently to the flow passage 60 so that the fluid flows along the flow passage 60 to deprive the heat of the eyeball 2 through the cornea-contact wall 61. The control member adjusts the suction speed of the pump 21 so that the flow passage 60 maintains its inner pressure suited to the curvature of the eyeball 2.

The fluid in the refrigerant device 65 is controlled at its temperature with the temperature of the fluid exhausted from the outlet hole 19 as a target temperature, so as to perform the laser treatment while cooling the eyeball 2.

### Advantages Derived from the Fourth Embodiment of the Invention

With the ophthalmic laser treatment device 1 running the fluid along the flow passage 60, the fluid deprives the heat of the eyeball 2 through the cornea-contact wall 61 so as to cool the eyeball 2 without making the fluid contact with the eyeball 2. This enables the operator to perform the laser treatment while cooling the eyeball 2 without considering an influence of the ingredients and streams of the fluid against the eyeball 2.

The control member adjusts the suction speed of the pump 21 so that the flow passage 60 maintains its inner pressure required for the cornea-contact wall 61 to represent the curvature suited to the curvature of the eyeball 2.

This enables the operator to fit the cornea-contact wall 61 snugly to the outer surface of the eyeball 2 without imposing an improper burden on the eyeball 2.

### Fifth Embodiment of the Invention

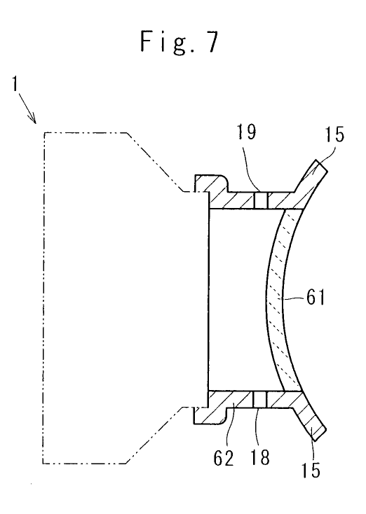

FIG. 7 shows a fifth embodiment of the invention in which the fluid-passage forming portion 62 is discrete.

The cornea-contact wall 61 is secured to an open end of the fluid-passage forming portion 62 in the proximity of the eyeball 2. The fluid-passage forming portion 62 is detachably mounted on the lens 3 and the lens holder 4.

Because the fluid-passage forming portion 62 is detachably arranged, it becomes possible to throw away the fluid-passage forming portion 62 each time after it is used once or several times, thus securing a good maintenance, high quality and sanitation preferable as a medical apparatus.

It is also possible to attach the spacer and the light-permeable member in various purposes to an existing ocular lens which is presently used widely to the ophthalmic laser treatment field.

### Sixth Embodiment of the Invention

### Structure of Sixth Embodiment

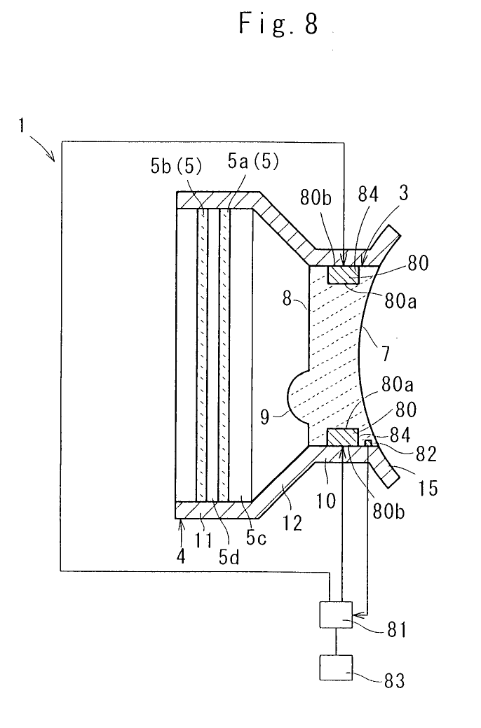

FIG. 8 shows a sixth embodiment of the invention in which the contact condition between the eyeball 2 and the lens 2 is generally identical to the contact condition as described in the first embodiment of the invention (FIG. 1).

In the sixth embodiment of the invention, the ophthalmic laser treatment device 1 has the lens 3 and the lens holder 4 which supports the lens 3. The partition wall 5 is provided which forms the hermetical space together with the lens 3. The lens 3 acts as the light-permeable member which is in contact with the eyeball 2 to introduce the laser beams to the eyeball 2.

As the same manner in the first embodiment of the invention, the lens 3 makes its concave surface side 7 bring into contact with the eyeball 2.

A peripheral open end of the holder cylinder 10 defines the engagement portion 15 at the side of the eyeball 2. The engagement portion 15 shapes its outer surface and the concave surface side 7 into the concave configuration in accordance with the outer surface of the eyeball 2.

In the ophthalmic laser treatment device 1 according to the sixth embodiment of the invention, there is provided a Peltier's coupler 80 attached to the lens 3. To the Peltier's coupler 80, a controller 81 is connected. To the controller 81, a thermal sensor 82 is provided which is attached to the lens 3. The Peltier's coupler 80 is a semi-conductor device which enables the operator to cool, heat and thermally control the lens 3 when the Peltier's coupler 80 is energized by the direct current. Upon energizing the Peltier's coupler 80 through a power source 83, the Peltier's coupler 80 produces a thermal difference between the two sides. The Peltier's coupler 80 absorbs the heat at a lower temperature side (refrigerant side 80a) and generates the heat at a higher temperature side (heat-radiant side 80b). The lower temperature side pushes up the heat toward the higher temperature side.

An outer periphery side of the lens 3 has two recessed portions 84 in which the Peltier's coupler 80 is placed. The Peltier's coupler 80 makes the refrigerant side 80a face the lens 3, and makes the heat-radiant side 80b face the inner surface of the holder cylinder 10.

The control member controls a polarity, an intensity of the direct current and an on-off switch of the Peltier's coupler 80 with the temperature detected by the thermal sensor 82 as a target temperature. This enables the operator to control a heat-absorbent amount of the Peltier's coupler 80 so as to adjust the temperature of the lens 3.

### Therapeutical Method According to the Sixth Embodiment of the Invention

With the lens 3 placed on the eyeball 2, the laser beams are released against the lens 3. During the release of the laser beams, the Peltier's coupler 80 is energized so that the refrigerant side 80a absorbs the heat from the lens 3 to cool the lens 3. The cooled lens 3 deprives the heat of the eyeball 2 during the release of the laser beams, so as to perform the laser treatment while cooling the eyeball 2.

### Advantages Derived from the Sixth Embodiment of the Invention

In the ophthalmic laser treatment device 1 according to the sixth embodiment of the invention, the Peltier's coupler 80 enables the operator to cool the lens 3 with the refrigerant side 80a placed to face the lens 3. The eyeball 2 loses the heat through the cooled lens 3 placed in contact with the eyeball 2.

With the eyeball 2 cooled while applying the laser beams to the eyeball 2, it is possible to prevent the complication which would be otherwise caused by the temperature rise of the eyeball 2 due to the laser beam application. When highly intensified laser beams are employed to enhance therapeutical effects, the refrigerant side 80a enables the operator to cool the lens 3 so as to avoid the complication.

With the use of the Peltier's coupler 80 as the refrigerant device, it is possible to secure a quick response to the temperature change so as to instantly cool the lens 3. The Peltier's coupler 80 makes the lens 3 immune to vibrations at the time of performing the laser treatment, as opposed to the case in which the fluid is used upon cooling the lens 3. The Peltier's coupler 80 has the quick response to the temperature change so as to perform the temperature control with a high precision.

In the sixth embodiment of the invention, the holder cylinder 10 may be shaped into a fin-like configuration to release the heat transmitted from the heat-radiant side 80b of the Peltier's coupler 80. As an alternative, a plurality of ventilation holes may be formed on the holder cylinder 10 to release the heat. The on-off switch may be operated manually without using the thermal sensor 80 and the controller 81. As a modification form of the eighth embodiment of the invention, the Peltier's coupler 80 may be placed outside the holder cylinder 10 with the refrigerant side 80a facing an outer surface of the holder cylinder 10.

### Seventh Embodiment of the Invention

### Structure of Seventh Embodiment

FIG. 9 shows a seventh embodiment of the invention in which the contact condition between the eyeball 2 and the lens 2 is generally identical to the contact condition as described in the first embodiment of the invention (FIG. 1). Described herein are mainly members other than the those of the sixth embodiment of the invention.

In the seventh embodiment of the invention, the ophthalmic laser treatment device 1 has the lens 3, an inner portion of which is hollowed out to form a reservoir 90 on the lens 3. An outer periphery of the lens 3 has a side opening 91, through which the fluid is supplied to the reservoir 90. The reservoir 90 retains the fluid by plugging side opening 91 with a rubber plug 92 so as to provide a hermetical space within the reservoir 90.

The Peltier's coupler 80 is mounted on the lens 3 with the refrigerant side 80a in contact with the fluid so as to cool the fluid in the reservoir 90. The Peltier's coupler 80 is placed at three locations with the circumferential intervals as 90 degrees as shown in FIG. 9(b).

### Therapeutical Method According to the Seventh Embodiment of the Invention

With the lens 3 placed on the eyeball 2, the laser beams are released against the lens 3. During the release of the laser beams, the Peltier's coupler 80 is energized so that the refrigerant side 80a absorbs the heat from the fluid to cool the fluid. The cooled fluid deprives the heat of the fluid to cool the lens 3. The cooled lens 3 deprives the heat of the eyeball 2 during the release of the laser beams, so as to perform the laser treatment while cooling the eyeball 2. Upon supplying the fluid to the reservoir 90, the rubber plug 92 may be unplugged, or a syringe needle may be used to pierce the rubber plug 92 inside the reservoir 90.

### Advantages Derived from the Seventh Embodiment of the Invention

In the ophthalmic laser treatment device 1 according to the seventh embodiment of the invention, the Peltier's coupler 80 enables the operator to cool the fluid within the reservoir 90 so as to deprive the heat of the lens 3. That is to say, the eyeball 2 loses the heat through the cooled lens 3 placed in contact with the fluid.

With the eyeball 2 cooled while applying the laser beams to the eyeball 2, it is possible to prevent the complication which would be otherwise caused by the temperature rise of the eyeball 2 due to the laser beam application. When highly intensified laser beams are employed to enhance therapeutical effects, the refrigerant side 80a enables the operator to cool the lens 3 so as to avoid the complication.

With the reservoir 90 provided in the inner portion of the lens 3, and the fluid cooled from within by the Peltier's coupler 80, the fluid enables the operator to efficiently cool the lens 3.

Such is the structure that the fluid is cooled within the lens 3, not outside the ophthalmic laser treatment device 1. This makes a therapeutical preparation ready by supplying the fluid to the reservoir 90 and energizing the on-off switch prior to using the ophthalmic laser treatment device 1. The facile preparation enables the operator to easily use the ophthalmic laser treatment device 1.

Instead of the structure that the Peltier's coupler 80 sets the refrigerant side 80a in contact with the fluid in the reservoir 90, the refrigerant side 80a may placed on an outer surface of the lens 3, by way of illustration, without making the refrigerant side 80 contact directly with the fluid, so long as the Peltier's coupler 80 can cool the fluid via the refrigerant side 80a.

### Modification Forms

As a modification form of the ninth embodiment of the invention, instead of using the control member (computer 22) including the pump 21 and the refrigerant device 65, the suction speed and the fluid temperature may be manually adjusted. A valve may be provided between the tank 20 and the inlet hole 18 (or between the outlet hole 19 and the pump 21) so as to adjust the fluid flow by controlling the opening degree of the valve.

The fluid source may be an intravenous drip sack containing ice and liquid. As the fluid-supply member, a siphon may be used to supply the fluid to the inner fluid passage 30, the outer side fluid passage 40 and the flow passages 50, 60.

The lens 3 (first embodiment), the refrigerant vessel 51 (fifth embodiment) and cornea-contact wall 61 (sixth embodiment) are in the shape of blind surface, however, these members may be partly perforated.

The Peltier's coupler may be incorporated into the ophthalmic laser treatment device 1 from the first to fifth embodiment of the invention.

The light-permeable member requires to permeate the light with the light-permeable member in contact with the eyeball 2 even if it has not a refractive property to collect the laser beams. Because the laser oscillator converges the laser beams to one point, the light-permeable member is not necessarily required to collect the laser beams. As the focusing member, a mirror-reflector device may be provided on the laser-incident side of the light-permeable member when the light-permeable member has not the refractive property. The mirror-reflector device brings the reflective laser beams into focus on any location of the eyeball 2.

### INDUSTRIAL APPLICABILITY

The ophthalmic laser treatment device 1 is useful upon performing the ophthalmic laser treatment in which the laser beams are therapeutically applied to the eyeball.

What is claimed is:

1. An ophthalmic laser treatment device comprising:
a light-permeable member provided to lead laser beams to an eyeball; and
a spacer member provided to maintain a predetermined space between said light-permeable member and said eyeball;
wherein said eyeball being cooled by introducing a fluid into said space so that said fluid comes in direct contact with said eyeball.

2. The ophthalmic laser treatment device according to claim 1, wherein said spacer is detachably mounted on said light-permeable member.

3. An ophthalmic laser treatment device comprising:
a light-permeable member provided to be in contact with an eyeball so as to lead laser beams to said eyeball; and
a fluid passage provided, through which a fluid flows to cool said eyeball;
wherein said fluid passage is located remote from said eyeball.

4. The ophthalmic laser treatment device according to claim 3, wherein said fluid passage is located inside said light-permeable member.

5. The ophthalmic laser treatment device according to any of claims 1-4, wherein a transparent partition wall is provided to form a space between said light-permeable member and a laser oscillator provided to release said laser beams.

6. The ophthalmic laser treatment device according to claim 3, wherein a transparent body is provided at one side of said light-permeable member, said one side being opposite to said eyeball, and a spacer is provided to form a space between said light-permeable member and said transparent body;
wherein said fluid passage is provided between said light-permeable member and said transparent body.

7. The ophthalmic laser treatment device according to claim 6, wherein said light-permeable member is formed by an elastic film of flexibility.

8. The ophthalmic laser treatment device according to claim 7, wherein a fluid-supply member is provided to supply said fluid, and a control member is provided to functionally control said fluid-supply member;
wherein said control member adjusts a flow of said fluid so that said light-permeable member conforms its configuration to a configuration of said eyeball.

9. The ophthalmic laser treatment device according to any of claims 6-8, wherein a transparent partition wall is provided to form a space between said transparent body and a laser oscillator provided to release said laser beams.

10. The ophthalmic laser treatment device according to any of claims 6-9, wherein said spacer and said light-permeable member are detachably mounted on said transparent body.

11. The ophthalmic laser treatment device according to any of claims 1-10, wherein a fluid-cooling member is provided to cool said fluid, and a control member is provided to control said fluid-cooling member;
wherein said fluid-cooling member works to adjust so that said fluid substantially conforms its temperature to a target temperature.

12. An ophthalmic laser treatment device comprising:
a light-permeable member provided to be in contact with an eyeball so as to introduce laser beams to said eyeball; and
a Peltier's coupler mounted on said light-permeable member;
wherein said light-permeable member is cooled by said Peltier's coupler so as to resultantly cool said eyeball.

13. The ophthalmic laser treatment device according to claim 12, wherein a reservoir is provided inside said light-permeable member to contain a fluid, and said Peltier's coupler works to cool said light-permeable member and said fluid contained inside said reservoir.

14. The ophthalmic laser treatment device according to claim 12 or 13, wherein a transparent partition wall is provided to form a space between said light-permeable member and a laser oscillator provided to release said laser beams.

15. The ophthalmic laser treatment device according to any of claims 1-14, wherein said light-permeable member is a lens member to collect said laser beams to bring said laser beams into focus on said eyeball.

16. The ophthalmic laser treatment device according to any of claims 1-15, wherein a focusing member is provided to bring the laser beams into focus on any position of said eyeball by refracting or reflecting said laser beams incident on said light-permeable member.
